(19) United States
(12) Patent Application Publication
(10) Pub. No.: US 2019/0223813 A1
(43) Pub. Date: Jul. 25, 2019

(54) CAPACITIVE ECG SENSING ELECTRONIC DISPLAYS AND RELATED METHODS

(71) Applicant: Intel Corporation, Santa Clara, CA (US)

(72) Inventor: Sean J. W. Lawrence, Bangalore (IN)

(21) Appl. No.: 16/370,917

(22) Filed: Mar. 30, 2019

(51) Int. Cl. A61B 5/00; G06F 3/041; G06F 3/044; A61B 5/044

(52) U.S. Cl. CPC A61B 5/7475; A61B 5/044; G06F 3/044; G06F 3/0416

(57) ABSTRACT

Capacitive ECG sensing electronic displays and related methods are disclosed herein. An example electronic device disclosed herein includes a display screen including a first electrode and a second electrode and a processor operatively coupled to the display screen. The processor is to cause the display screen to operate in a first display screen mode to detect a touch input from a user on the display screen. The processor is to cause the display screen to switch from operating in the first display screen mode to operating in a second display screen mode. The first electrode and the second electrode are to generate signal data indicative of electrocardiogram data for the user when the display screen is operating in the second display screen mode.

### FIELD OF THE DISCLOSURE

This disclosure relates generally to electronic displays and, more particularly, to capacitive electrocardiogram (ECG) sensing electronic displays and related methods.

### BACKGROUND

Electrocardiogram (ECG) sensors measure electrical activity of a heart of a subject. ECG signal data can be generated by capacitive electrodes that do not require a use of a conductive gel to reduce skin resistance. Some capacitive ECG electrodes can detect ECG signals from a subject through an object disposed between the electrode and skin of the user, such as clothing.

### BRIEF DESCRIPTION OF THE DRAWINGS

FIG. 1 illustrates an example system constructed in accordance with teachings of this disclosure and including an example electronic display screen and a touch controller for controlling the electronic display, where the electronic display screen is operating in a first display screen mode at a touch screen.

FIG. 2 illustrates the example display screen of FIG. 1 operating in a second display screen mode as an ECG sensor.

FIG. 3 illustrates an example capacitor of the example display screen of FIGS. 1 and 2.

FIG. 4 illustrates an example personal computing device including the example display screen of FIGS. 1 and 2, where the display screen is operating in the first display screen mode.

FIG. 5 illustrates an example personal computing device including the example display screen of FIGS. 1 and 2, where the display screen is operating in the second display screen mode.

FIG. 6 is a block diagram of an example implementation of the touch controller of FIG. 1.

FIG. 7 is a flowchart representative of example machine readable instructions which may be executed to implement the example touch controller of FIGS. 1, 2, and/or 6.

FIG. 8 is a block diagram of an example processing platform structured to execute the instructions of FIG. 7 to implement the example touch controller of FIGS. 1, 2, and/or 6.

The figures are not to scale. Instead, the thickness of the layers or regions may be enlarged in the drawings. In general, the same reference numbers will be used throughout the drawing(s) and accompanying written description to refer to the same or like parts. Although the figures show layers and regions with clean lines and boundaries, some or all of these lines and/or boundaries may be idealized. In reality, the boundaries and/or lines may be unobservable, blended, and/or irregular.

Descriptors “first,” “second,” “third,” etc. are used herein when identifying multiple elements or components which may be referred to separately. Unless otherwise specified or understood based on their context of use, such descriptors are not intended to impute any meaning of priority, physical order or arrangement in a list, or ordering in time but are merely used as labels for referring to multiple elements or components separately for ease of understanding the disclosed examples. In some examples, the descriptor “first” may be used to refer to an element in the detailed description, while the same element may be referred to in a claim with a different descriptor such as “second” or “third.” In such instances, it should be understood that such descriptors are used merely for ease of referencing multiple elements or components.

### DETAILED DESCRIPTION

Electrocardiogram (ECG) sensors measure electrical activity of a heart of a subject. ECG signal data can be generated by capacitive electrodes that do not require a use of a conductive gel to reduce skin resistance and that are capable of detecting ECG signals from a subject through an object such as clothing. In particular, a capacitor is formed between the ECG electrode and skin of the user within a particular distance of the electrode (e.g., within 1 mm). Electrical activity generated by the user's heart and detectable via the user's skin affects the electrical charge distribution of the electrode, or self-capacitance of the electrode. Signal data indicative of changes in self-capacitance of the electrode can be used to derive the user's ECG data. For example, first signal data can be collected from a first electrode that forms a first capacitator with a first portion of the user's body (e.g., the user's right thumb) and second signal data can be collected from a second electrode that forms a second capacitator with a second portion of the user's body corresponding to the first portion of the user's body (e.g., the user's left thumb). The ECG data for the user can be calculated by taking the difference between the first signal data and the second signal data.

Some known personal computing devices include ECG sensors to obtain ECG data while the user is interacting with the device. For example, some known personal computing devices include ECG sensors located at the palm rests of, for instance a laptop keyboard. Some known personal computing device(s) include ECG sensors located on key cap(s) of a keyboard and/or at the hinge(s) of the device(s). However, such incorporation of the ECG sensors into the personal computing devices typically involves additional hardware and/or modifications to the design (e.g., form factor) of the device(s).

Disclosed herein are example display screens that include capacitive ECG electrodes to selectively generate ECG signal data for a user interacting with the display screen. Examples disclosed herein include a touch controller operatively coupled to the display screen. The touch controller causes the display screen to operate in a first mode as a touch screen that receives touch inputs from the user as the user interacts with digital content displayed via the display screen. In examples disclosed herein, the touch controller selectively instructs the display screen to operate in a second mode in which the ECG electrodes are activated to generate ECG data for the user when the user touches the portion(s) of the display screen including the ECG electrodes.

Example display screens disclosed herein includes drive lines (e.g., electrical traces, metal wires) that transmit drive signals received from a drive signal generator when the display screen is operating in the touch screen mode and sense lines (e.g., electrical traces, metal wires) to detect the signals carried by the drive lines. When the user touches the display screen, a voltage drop occurs at the location where the user touched the display screen 102 due to electrical charges transmitted by, for example, the user's fingertip. The voltage changes are detected by the sense lines and used by the touch controller to identify touch events on the display screen.

In examples disclosed herein, the ECG electrodes of the display screen include at least some of the drive lines of the display screen. To generate ECG data, the touch controller instructs the drive signal source to refrain from transmitting drive signals to the drive lines of the display screen. When the user places his or her finger(s), palm(s), etc. on the portions of the display screen including the ECG electrodes, the drive lines that form the electrodes detect changes in self-capacitance due to electrical charges on the skin of the user generated by the user's heart activity. The difference between signal data generated from two ECG electrodes of the display screen is used by the touch controller to determine the user's ECG signal data. The ECG signal data can be analyzed to, for instance, monitor the user's health.

When the ECG signal data has been collected from the user, the example touch controller instructs the driver signal source to resume transmitting drive signals to the drive lines of the display screen, thereby reactivating the touch screen functionalities of the display screen. Thus, example display screens disclosed herein incorporate ECG-sensing capabilities without significant additions and/or modifications to the hardware and/or form factor of the personal computing device that includes the display screen. Rather, examples disclosed herein provide for dual use of the drive lines of the display screen in enabling touch functionalities of the display screen and in detecting ECG signal data based on changes in capacitance.

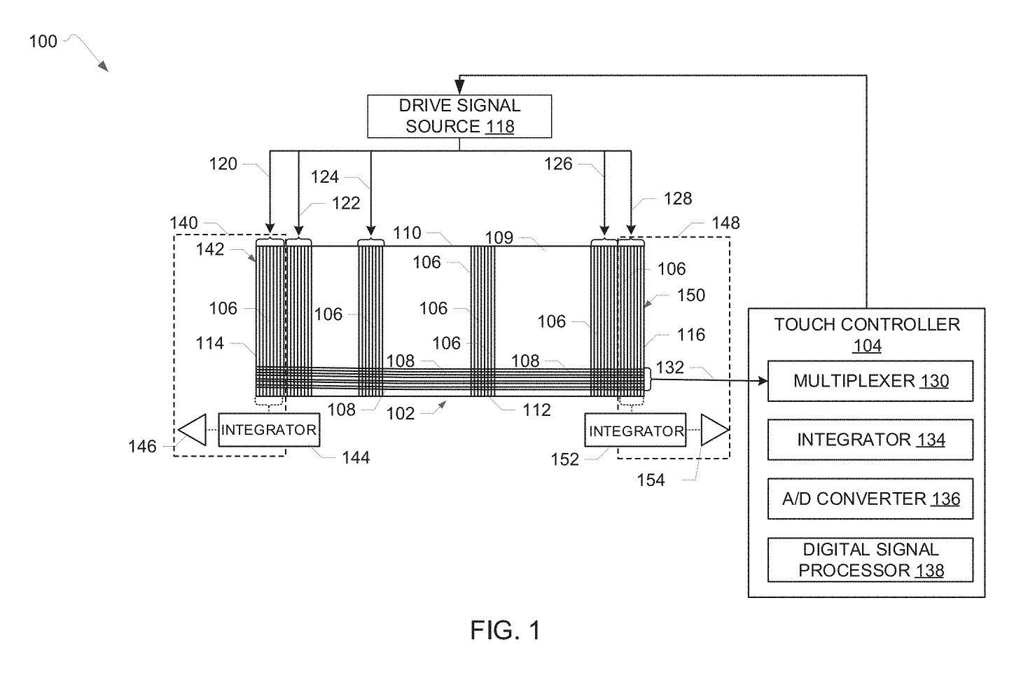

FIG. 1 illustrates an example system 100 constructed in accordance with teachings of this disclosure including an electronic display screen 102 and a touch controller 104 for controlling operation of the display screen 102. In the example system 100 may be implemented in a personal computing device, such as a laptop or electronic tablet. The example display screen 102 enables a user of the personal computing device to view digital data via user interface(s) and/or to provide input(s) to interact with application(s) on the device. In particular, the example display screen 102 of FIG. 1 is a touch screen that enables the user to interact with the data presented on the display screen 102 by touching the screen with one or more fingers of a hand of the user and/or with a stylus based on changes in mutual capacitance detected by electrodes of the display screen 102 when the user and/or stylus contact the display screen 102. As disclosed herein, the example touch controller 104 of FIG. 1 selectively controls operation of the display screen 102 between a first mode in which the display screen 102 is a touch screen that enables the user to interact with the device via touch gesture(s) on the screen and a second mode in which electrodes of the display screen 102 are activated to generate ECG signal data from the user (the terms “user” and “subject” are used interchangeably herein and both refer to a biological creature such as a human being). As discussed herein, the example touch controller 104 can be implemented by software executed on a processor of the personal computing device including the display screen 102. In some examples, one or more components of the touch controller 104 are implemented by a cloud-based device.

The example display screen 102 of FIG. 1 is a capacitive display screen including a plurality of drive lines 106 and a plurality of sense lines 108. The drive lines 106 and the sense lines 108 include electrical traces or wires (e.g., metal) on a conductive substrate 109 of the display screen 102. The conductive layer including the drive lines 106 and the sense lines 108 is disposed between a glass substrate and a protective cover, which can include glass and/or a protective coating. For illustrative purposes, the glass substrate and the protective cover are not shown in FIG. 1.

As illustrated in FIG. 1, the drive lines 106 extend between a first edge 110 of the display screen 102 and a second edge 112 of the display screen 102 opposite the first edge 110. The sense lines 108 extend between a third edge 114 of the display screen 102 and a fourth edge 116 of the display screen 102 opposite the third edge 114. In the example of FIG. 1, the sense lines 108 are perpendicular to the drive lines 106 to form a grid pattern.

The example display screen 102 can include additional or fewer drives lines 106 and/or sense lines 108 than shown in FIG. 1. For example, although for illustrative purposes, the sense lines 108 of FIG. 1 are shown as extending across a portion of the display screen 102 (e.g., the bottom portion when the display screen 102 is oriented as in FIG. 1), the sense lines 108 can extend across additional areas of the display screen 102 such that the grid pattern is formed between the first and second edges 110, 112 of the display screen. Also, the spacings between the respective drive lines 106 can differ from the spacings shown in FIG. 1. Similarly, the spacings between the respective sense lines 108 can differ from the spacings shown in FIG. 1. Also, the display screen 102 can have a different size and/or shape than the example shown in FIG. 1.

In the example system 100 of FIG. 1, the touch controller 104 is communicatively coupled to a drive signal source 118 (e.g., a generator). When the display screen 102 is functioning as a user touch screen, as shown in FIG. 1, the example touch controller 104 instructs the drive signal source 118 to emit drive signals (e.g., timed pulses) that are carried by the drive line(s) 106, as represented by the lines 120, 122, 124, 126, 128 in FIG. 1. The sense line(s) 108 detect the current carried by the drive line(s) 106 at the location(s) at which the respective sense line(s) 108 intersect with the drive line(s) 106. The sense line(s) 108 generate signal data based on the detected current and transmit the signal data to a multiplexer 130 of the touch controller 104, as represented by line 132 in FIG. 1.

When, for example, the user's fingertip touches the display screen 102, a voltage drop occurs at the location where the user's fingertip touches the display screen 102. The change in voltage is detected by the sense line(s) 108. The sense line(s) 108 of the display screen 102 transmit signal data indicating the change in voltage to the multiplexer 130 of the touch controller 104. The multiplexer 130 forwards the signals received from the various sense lines 108 to an integrator 134 of the touch controller 104. The integrator 134 performs an integration of the signal data to generate an output voltage indicative of the change(s) in voltage detected by the sense line(s) 108 over time. The example touch controller 104 includes an analog-to-digital (A/D) converter 136 to generate a digital signal that is transmitted to a digital signal processor 138. The digital signal processor 138 interprets the signal data to detect touch event(s) at particular locations on the display screen 102 where the voltage change(s) were detected by the sense line(s) 108. The touch controller 104 communicates the touch event(s) to, for example, a processor the personal computer device that executes software to interpret and output response(s) based on the touch event(s).

Thus, the example display screen 102 operates in a first mode as a touch screen that enables a user to interact with data presented via the display screen 102 by providing touch inputs on the screen. As mentioned above, in some examples, the touch controller 104 causes the display screen 102 to act in a second mode in which at least some of the drive lines 106 are converted to electrodes that measure changes in self-capacitance when the user touches the display screen 102. The changes in self-capacitance are used by the touch controller 104 to derive ECG data for the user.

The example display screen 102 of FIG. 1 includes a first ECG sensing electrode 140, or first means for sensing EGC data. The first ECG sensing electrode 140 includes a first drive line set 142 including one or more of the drive lines 106, a first integrator 144, and a first amplifier 146. The display screen 102 includes a second ECG sensing electrode 148, or first means for sensing EGC data. The second ECG sensing electrode 148 includes a second drive line set 150 including one or more of the drive lines 106 different from the drive line(s) 106 of the first drive line set 142, a second integrator 152, and a second amplifier 154. As shown in FIG. 1, the first ECG sensing electrode 140 is proximate to the third edge 114 of the display screen 102 and the second ECG sensing electrode 148 is proximate to the fourth edge 116 of the display screen 102. To collect ECG data, a user places, for example, a thumb of his or her left hand on the display screen 102 proximate to the third edge 114 and, thus, the first ECG sensing electrode 140, and a thumb of his or her right hand on the display screen 102 proximate to the fourth edge 116 and, thus, the second ECG sensing electrode 148. The user can use other portion(s) of this body to contact the display screen 102 (e.g., palms, wrists, other fingers).

In the example of FIG. 1, the first and second ECG sensing electrodes 140, 148 are in an inactive state when the display screen 102 is operating in the first operational mode as a touch screen. To cause the display screen 102 to operate in the second operational mode as an ECG sensor, the touch controller 104 selectively activates the first and second ECG sensing electrodes 140, 148 by converting the drives lines 106 in the first drive line set 142 of the first ECG sensing electrode 140 and the drive lines 106 in the second drive line set 150 of the second ECG sensing electrode 148 from drive signal transmitting lines to capacitive electrodes. The touch controller 104 activates the integrator(s) 144, 152 and the amplifier(s) 146, 154 (e.g., by generating instruction(s) for the integrator(s) 144, 152 and the amplifier(s) 146, 154 to receive data from the drive line(s) 106). In some examples, the touch controller 104 activates the ECG sensing electrodes 140, 148 after detecting that the user has touched one or more areas of the display screen 102 proximate to the third and fourth edges 114, 116 (and, thus, proximate to one or more of the ECG sensing electrodes 140, 148) for a predetermined period of time.

When the touch controller 104 of FIG. 1 determines that first and second ECG sensing electrodes 140, 148 should be activated, the touch controller 104 refrains from instructing the drive signal source 118 to generate and transmit drive signals to the drive lines 106 of the display screen 102. Thus, touch screen functionality of the display screen 102 is temporarily disabled when the first and second ECG sensing electrodes 140, 148 are activated. By instructing the drive signal source 118 to refrain from transmitting drive signals to the drive lines 106 (including the drive lines 106 of the display screen 102 that are not a part of the first drive line set 142 and the second drive line set 148), the sensitivity of the first and second ECG sensing electrodes 140, 148 in detecting small changes in capacitance is increased due to less noise. As a result, the ECG sensing electrodes 140, 148 can more accurately detect ECG signals.

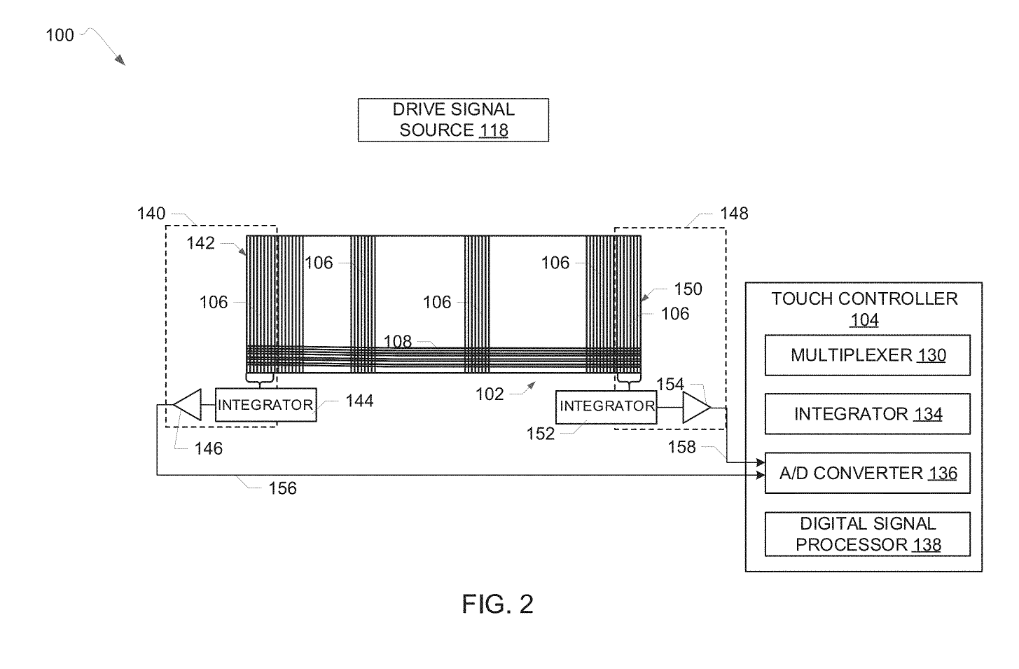

FIG. 2 illustrates the example system 100 including the display screen 102 and the touch controller 104, where the display screen 102 is operating in the second display screen mode as an ECG sensor. As mentioned above, the touch controller 104 refrains from sending instructions to the drive signal source 118 when the display screen 102 is operating in the second mode. Thus, because the drive lines 106 do not transmit signals, the sense lines 108 do not transmit signal data to the multiplexer 130 measuring the current carried by the drive line(s) 106. Therefore, the touch functionality of the display screen 102 is disabled.

To measure ECG data, the user touches the portion of the display screen 102 including the first ECG sensing electrode 140 with, for example, his or her left thumb, and the portion of the display screen 102 including the second ECG sensing electrode 148 with a corresponding body part on the right side, such as his or her right thumb. In the example of FIG. 2, when the user touches the portion of the display screen 102 including the first ECG sensing electrode 140, a capacitator is formed between the skin of the user and the drive lines 106 of the first drive line set 142. Electrical activity generated by the heart of the user affects the electric charge stored by the drive line(s) 106 of the first drive line set 142 of the first ECG sensing electrode 140. The drive lines(s) 106 of the first drive line set 142 generate signal data in response to the change in electric charge due to the user's heart activity. The signal data is transmitted to the first integrator 144 of the first ECG sensing electrode 140. The first integrator 144 of the first ECG sensing electrode 140 integrates the signal data generated by the respective drive lines 106 of the first drive line set 142. The signal output by the first integrator 144 is transmitted to the first amplifier 146 of the first ECG sensing electrode 140, as represented by line 156 in FIG. 2. The first amplifier 146 amplifies the signal and transmitted the amplified signal to the A/D converter 136 of the touch controller 104.

Similarly, when the user touches the portion of the display screen 102 including the second ECG sensing electrode 148, a capacitator is formed between the skin of the user and the drive lines 106 of the second drive line set 150. Changes in capacitance due to the electrical activity of the heart of the user are detected by the drive line(s) 106 of the second drive line set 150 and the corresponding signal data is transmitted to the second integrator 152 of the second ECG sensing electrode 148. The second integrator 152 of the second ECG sensing electrode 148 integrates the signal data generated by the respective drive lines 106 of the second drive line set 150 and outputs a signal for amplification by the second amplifier 154 of the second ECG sensing electrode 148. The second amplifier 154 transmits the amplified signal to the A/D converter 136 of the touch controller 104, as represented by line 158 in FIG. 2.

The A/D converter 136 converts the analog signal data from the first ECG sensing electrode 140 and the second ECG sensing electrode 148 to digital signal data for analysis by the digital signal processor 138 of the touch controller 104. The digital signal processor 138 generates ECG signal data by calculating the difference between the signal data from the first ECG sensing electrode 140 and the signal data from the second ECG sensing electrode 148. In some examples, the digital signal processor 138 of FIG. 1 analyzes the ECG signal data to determine characteristics of the heart activity of the user, detect abnormalities, etc. In some examples, the digital signal processor 138 transmits the ECG signal data for analysis by other user application(s) installed on the personal computing device that includes the display screen 102 or on other user device(s) and/or cloud-based device(s).

In some examples, the touch controller 104 instructs the display screen 102 to switch from operating the second ECG-sensing mode to the first touch screen mode after a predetermined period of time and/or based on one or more trigger events. For example, the touch controller 104 can instruct the drive signal source 118 to resume generating and transmitting drive signals to the drive lines 106 when the user removes his or her finger(s) from the portion(s) of the display screen 102 including the first ECG sensing electrode 140 and/or the second ECG sensing electrode 148. In some examples, the touch controller 104 instructs the drive signal source 118 to resume generating and transmitting drive signals to the drive lines 106 based on other trigger events corresponding to, for instance, the calculation of the ECG signal data, the presentation of the ECG signal for display via the display screen 102, etc. When the touch controller 104 instructs the drive signal source 118 to resume transmitting the drive signals, the drive lines 106 that form the first and second drive line sets 142, 150 of the ECG sensing electrodes 140, 148 return to transmitting drive signals for measurement by the sense lines 108 in connection with detecting touch events on the display screen 102. The touch controller 104 instructs the integrators 144, 152 and the amplifiers 146, 154 of the respective ECG sensing electrodes 140, 148 to refrain from processing signal data associated with the drive lines 106. Thus, the touch controller 104 of FIGS. 1 and 2 selectively controls activation of the ECG sensing electrodes 140, 148 and, thus, operation of the display screen 102 as a touch screen or as an ECG sensor.

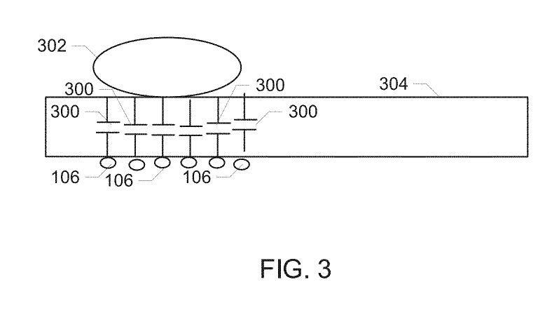

FIG. 3 illustrates example capacitors 300 formed between respective drive lines 106 of the example display screen 102 of FIGS. 1 and 2 and a finger 302 of a user of the display screen 102. The drive lines 106 shown in FIG. 3 can be included in first drive line set 142 of the first ECG sensing electrode 140 or the second drive line set 150 of the second ECG sensing electrode 148 of FIGS. 1 and 2.

In the example of FIG. 3, a glass substrate 304 is disposed over the drive lines 106 to form a protective cover between the drive lines 106 and the user. The glass substrate 304 also serves as a dielectric for the capacitors 300 formed between the user's finger 302 and the respective drive lines 106 of the display screen 102. The capacitance for one of the drive wires 106 of FIG. 3 can be calculated using the following equation:

$$C = \frac{2 π l {ϵ}_{0} {ϵ}_{r}}{{cosh}^{- 1} \frac{h}{r}} , \qquad ( Equation 1 )$$

where ε₀ is the permittivity of air (e.g., 8.854×10⁻¹²F·m), εᵣ is the dielectric constant of glass, r is the radius of the drive line wire, l is the length of the drive line wire, and h is the thickness of the glass 302.

For example, for one of the drive lines 106 of FIG. 3 having a radius of 5 μm and a length of 100 mm and where the glass 302 has a thickness of 1 mm and dielectric constant from 3-10, the capacitance of the drive line 106 is approximately 10 pF. In some examples, a sensor with a capacitance of 50 pF can detect ECG signals thorough, for example, clothing. In the examples of FIGS. 1-3, the first ECG sensing electrode 140 and the second ECG sensing electrode 148 including at least five drive lines 106 can be used to detect ECG signals from the user (e.g., 5 drive lines having a capacitance of 10 pF for a total capacitance of 50 pF).

To increase capacitance and, thus, the ECG signal detection capabilities of the ECG sensing electrodes 104, 148, the number of drive lines 106 that are used for ECG sensing can be increased. In some examples, a spacing between each of the drive lines 106 is minimized to increase the number of drive lines 106 in the drive line set(s) 142, 150 and, thus, the number of capacitors 300 formed between the user's finger 302 and the drive lines 106. In some examples, the number and/or spacing of the drive lines 106 can be based on an average size of, example, a human finger.

In some examples, conductive plates (e.g., metal plates) may be used with the ECS sensing electrode(s) 140, 148 in addition to or instead of the drive lines 106 to increase capacitance and ECG signal fidelity. Conductive plates may be used in examples where loss of touch display screen capabilities at the portions of the display screen that include the conductive plates when the display screen 102 is operating in the touch screen mode does not substantially affect use of the display screen. For example, conductive plates can be used in display screens that are dedicated to collecting ECG data and/or larger display screens in which a portion of the display screen real estate can be designated for ECG data collection without substantially reducing an area of the display screen available for the presentation of data.

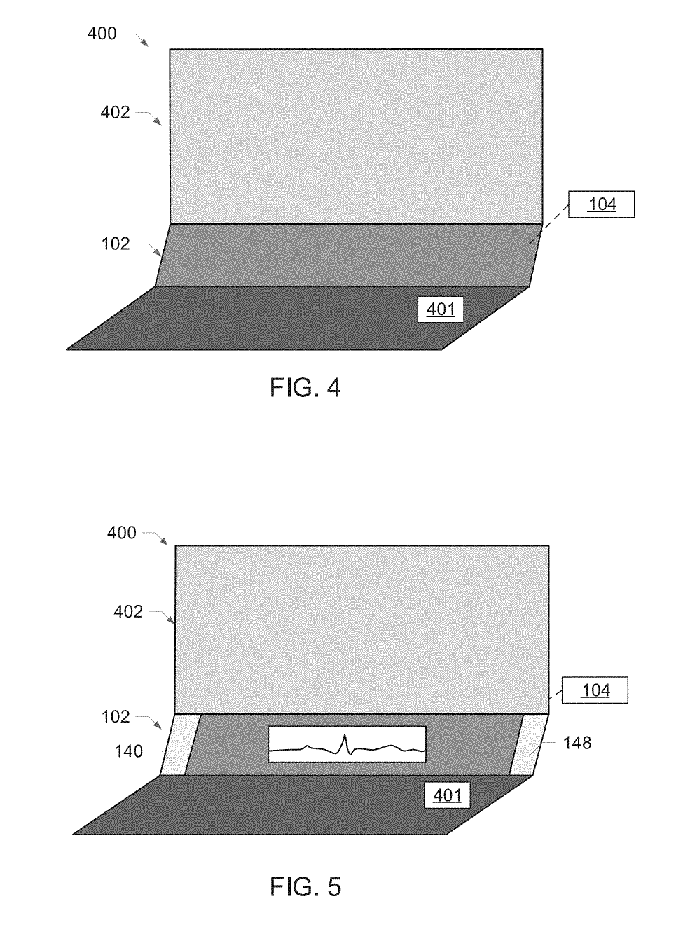

FIG. 4 illustrates an example personal computing device 400 including the example ECG-sensing display screen 102 of FIGS. 1 and 2. In FIG. 4, the example personal computing device 400 is a laptop including a processor 401. In addition to the ECG-sensing display screen 102, the example personal computing device 400 includes a second touch display screen 402 to display data and receive user touch input(s) for interacting with the data. In some examples, the second display screen 402 serves as the primary display screen for viewing and interacting with data displayed via the second display screen 402. When operating in the first mode as a touch screen, the ECG-sensing display screen 102 can display digital data in addition to the data displayed via the second display screen 402. The data displayed via the display screens 102, 402 of the personal computing device 400 of FIG. 4 can be associated with software application(s) executed by the processor 401 of the personal computing device 400. Thus, in the example of FIG. 1, both of the display screens 102, 402 operate as touch screens.

FIG. 5 illustrates the example personal computing device 400 of FIG. 4 including the display screen 102 of FIGS. 1 and 2 in the second operational state in which the first and second ECG sensing electrodes 140, 148 are activated. As disclosed above, the touch controller 104 (FIGS. 1 and 2) selectively activates the first and second ECG sensing electrodes 140, 148 to generate ECG data for a user touching the areas of the display screen 102 including the electrodes 140, 148. In the example of FIG. 5, the second display screen 402 continues to function as a touch screen for receiving user touch input(s). As discussed above, when the ECG-sensing display screen 102 is in the ECG sensing mode, the touch screen functionality of the display screen 102 is disabled with respect to receiving user touch input(s) to interact with data displayed via the display screen 102. Thus, in the example of FIG. 5, the display screen 102 is acting as an ECG sensor and the second display screen 402 is acting as a touch screen.

In some examples, one or more of the display screens 102, 402 display health data associated with the ECG signal, such as an ECG signal graph 500, heart rate data, etc. As discussed herein, the graphical health data can be generated by one or more software applications based on the ECG data generated by the first and second ECG sensing electrodes 140, 148.

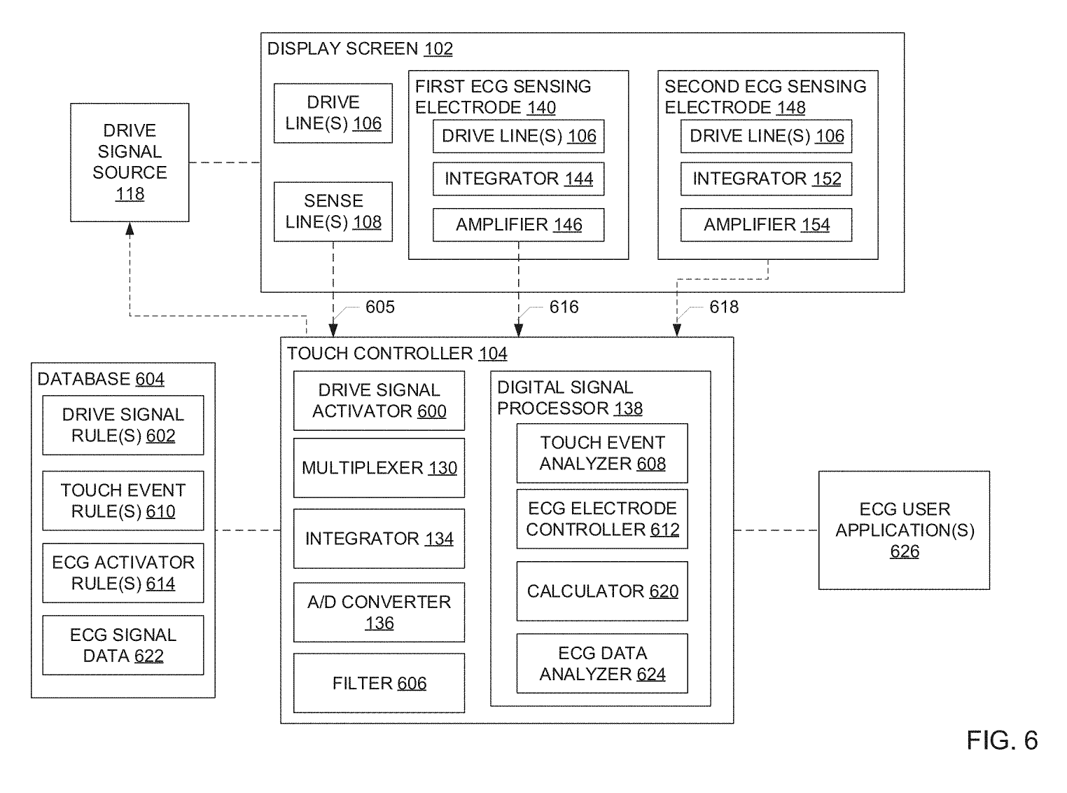

FIG. 6 is a block diagram of an example implementation of the example touch controller 104 of FIGS. 1 and 2. As mentioned above, the example touch controller 104 is constructed to cause a display screen (e.g., the display screen 102 of FIGS. 1 and 2) to operate in (1) a first mode in which the display screen functions as a touch screen to receive touch input(s) from a user to enable the user to interact with data displayed via the display screen, and (2) a second mode in which the display screen 102 acts as an ECG sensor by obtaining ECG signal data from the user via ECG sensing electrodes (e.g., the ECG sensing electrodes 140, 148). In the example of FIG. 6, the touch controller 104 is implemented by one or more processors of a personal computing device that includes the display screen 102 (e.g., the processor 401 of the personal computing device 400 of FIGS. 4 and 5). In some examples, some of the user touch event analysis and/or ECG data analysis implemented by the touch controller 104 via a cloud-computing environment and one or more other parts of the analyses is implemented by a processor of the personal computing device.

In some examples, the location(s) at which the analysis is performed by the touch controller 104 is based on the whether the analysis is to be performed in substantially real-time as the signal data is being generated or at a later time. For example, signal data generated by the sense lines 108 of the display screen 102 may be analyzed by the touch controller 104 at the processor 401 of the personal computing device 400 to detect touch event(s) on the display screen 102 in real-time. In other examples, analysis of the ECG signal data may be performed at a later time via a cloud-computing environment.

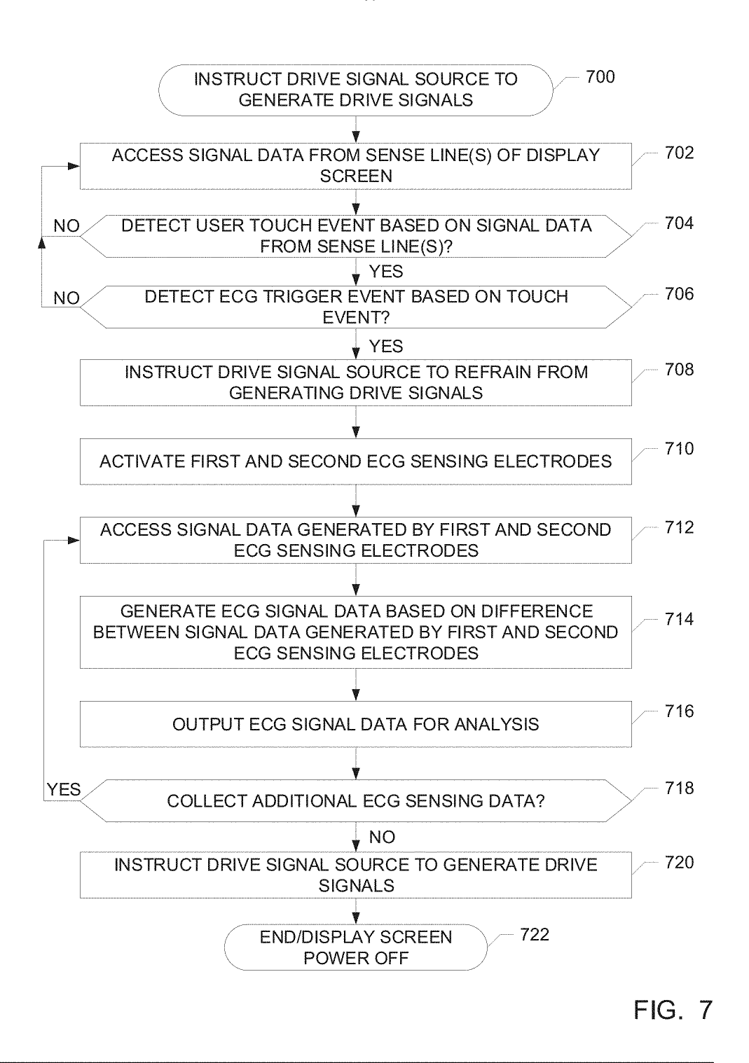

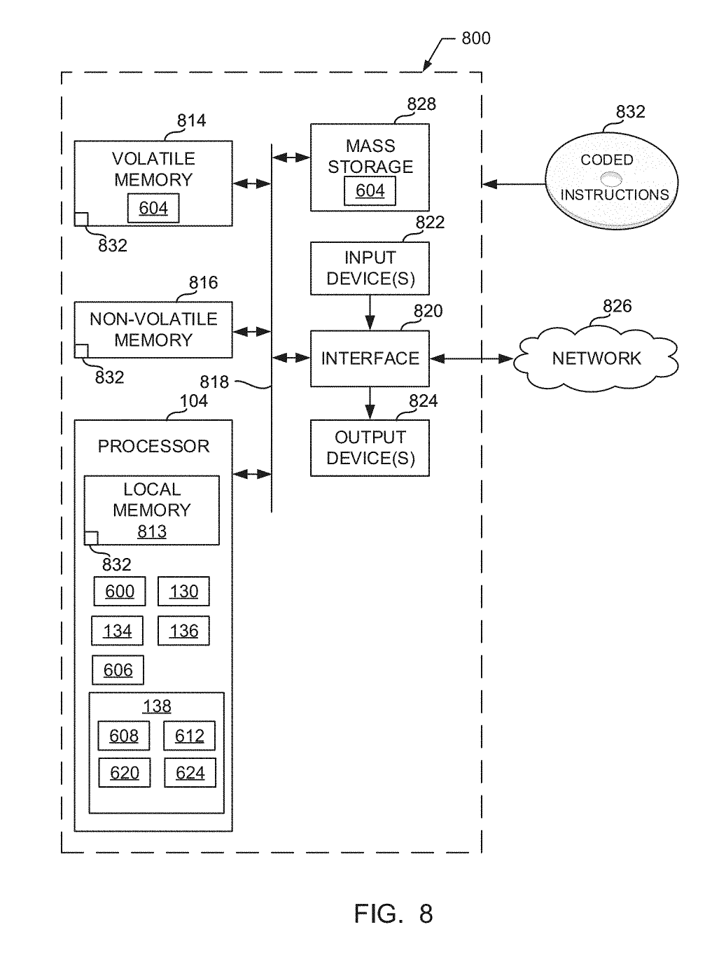

The example touch controller 104 selectively controls the operation of the display screen 102 between the first operation mode (i.e., the touch screen mode) and the second operation mode (i.e., the ECG sensing mode) by controlling transmission of the drive signals to the drive lines 106 of the display screen 102. The example touch controller 104 includes a drive signal activator 600. The drive signal activator 600 selectively instructs the drive signal source 118 to generate drive signals that are transmitted to the drive lines 106 of the display screen 102. Thus, the drive signal activator 600 provides means for causing the display screen 102 to operate in the first operation mode as a touch screen based on the transmission of the drive signals to the drive lines 106. The means for causing the display screen 102 to operate in the first operation mode as a touch screen may be implemented by a processor such as the processor of FIG. 8 executing instructions such as the instructions of FIG. 7.

In the example of FIG. 1, the drive signal activator 600 instructs the drive signal source 118 to generate the drive signals based on drive signal rule(s) 602 stored in a database 604. In some examples, the touch controller 104 includes the database 604. In other examples, the database 604 is located external to the touch controller 104 in a location accessible to the touch controller 104 as shown in FIG. 6. The example drive signal rule(s) 602, which can be defined by user input(s), can define, for example, the frequency at which the drive signal source 118 should emit drive signals, the number of drive signal pulses, etc.

As disclosed above, the sense line(s) 108 generate signal data 605 indicative of voltage drops with respect to the current flowing through the drive line(s) 106 when the user touches the display screen 102. The signal data 605 from the sense line(s) 108 can be stored in the database 604. The example touch controller 104 includes the multiplexer 130, which receives the signal data 605 from the sense line(s) 108 and outputs the signal data via an output line to the integrator 134 of the touch controller 104. The example touch controller 104 includes the A/D converter 136 to convert the analog signal data 605 from the sense line(s) 108 to digital data for processing by the digital signal processor 138 of the example touch controller 104.

In some examples, the touch controller 104 of FIG. 6 includes a filter 606 (e.g., a band pass filter). The example filter 606 substantially removes noise from the signal data 605 generated by the sense line(s) 108 before the data is provided to the digital signal processor. For example, the filter 606 can filter the signal data 605 to remove data indicative of voltage changes due to the user touching the display screen 102 at, for example, locations(s) on the display screen that are not associated with receiving input(s) (e.g., a user touch on a background image displayed via the display screen).

As mentioned above, the signal data 605 generated by the sense line(s) 108 is analyzed by the digital signal processor 138 of the touch controller 104 to identify touch event(s) on the display screen 102. The example digital signal processor 138 of FIG. 6 includes a touch event analyzer 608, or means for detecting touch on the display screen 102. The means for detecting touch may be implemented by a processor such as the processor of FIG. 8 executing instructions such as the instructions of FIG. 7.

The touch event analyzer 608 analyzes the signal data 605 from the sense line(s) 108 to identify touch event(s) based on one or more touch event rule(s) 610. The touch event rule(s) 610, which can be defined by user input(s), define threshold value(s) or range(s) for the voltage drop(s) measured by the sense line(s) 108 that are indicative of user touch event(s). In some examples, the touch event rule(s) 610 include a mapping of the sense line(s) 108 relative to the drive lines(s) 106 of the display screen 102. The mapping can be used by the touch event analyzer 608 to determine the location of the touch event(s) on the display screen 102.

The example touch controller 104 of FIG. 6 includes an ECG electrode controller 612, or means for selectively activating the first and second ECG sensing electrodes 140, 148 of the display screen 102. The means for selectively activating may be implemented by a processor such as the processor of FIG. 8 executing instructions such as the instructions of FIG. 7. The example ECG electrode controller 612 determines whether the ECG sensing electrodes 140, 148 should be activated based on one or more ECG activator rules 614. The ECG activator rule(s) 614 can be defined by user input(s) and stored in the database 604. The ECG activator rule(s) 614 define criteria that, when satisfied, cause the ECG electrode controller 612 to generate instructions that cause the display screen 102 to switch from operating in the first touch screen operational state to the second ECG sensing mode. In some examples, the criteria defined by the ECG activator rule(s) 614 are based on touch event(s) detected by the touch event analyzer 608.

For example, the ECG activator rule(s) 614 include a rule that the ECG electrode controller 612 should activate the ECG sensing electrodes 140, 148 if the touch event analyzer 608 detects that the user has touched the display screen 102 simultaneously at two different locations for a predetermined period of time (e.g., using his or her right and left fingers). ECG activator rule(s) 614 can include a rule that the ECG electrode controller 612 should activate the ECG sensing electrodes 140, 148 when the touch event analyzer 608 determines that the user has touched the regions of the display screen 102 that include the ECG sensing electrodes 140, 148 (e.g., proximate to the third and fourth edges 114, 116 of the display screen of FIG. 1) for predetermined period of time.

When the ECG electrode controller 612 determines that the criteria for activating the ECG sensing electrodes 140, 148 as defined by the ECG activator rule(s) 614 has been satisfied, the ECG electrode controller 612 communicates with the drive signal activator 600 to cause the drive signal source 118 to refrain from transmitting the drive signals to the drive line(s) 106 of the display screen 102. For example, based on instructions from the ECG electrode controller 612, the drive signal activator 600 can refrain from sending instructions to the drive signal source 118 to generate the drive signals. Also, the ECG electrode controller 612 transmits instructions to activate the first and second integrators 144, 152 and the first and second amplifiers 146, 154 of the first and second ECG sensing electrodes 140, 148. Thus, the display screen 102 switches from operating in the first touch screen mode to operating in the second ECG-sensing mode.

As disclosed above, the drive line(s) 106 of the first ECG sensing electrode 140 (e.g., the drive line(s) 106 of the first drive line set 142) generate first signal data 616 in response to changes in capacitance detected at the drive line(s) 106 due to the user's electrical heart activity. The first signal data 616 generated by the first ECG sensing electrode 140 is transmitted to the A/D converter 136 of the touch controller 104 (e.g., after integration by the first integrator 144 and amplification by the first amplifier 146 of the first ECG sensing electrode 140). Also, the drive line(s) 106 of the second ECG sensing electrode 148 (e.g., the drive line(s) 106 of the second drive line set 150) generate second signal data 618 in response to changes in capacitance detected at the drive line(s) 106 due to the user's electrical heart activity. The second signal data 618 generated by the second ECG sensing electrode 148 is transmitted to the A/D converter 136 of the touch controller 104 (e.g., after integration by the first integrator 152 and amplification by the first amplifier 154 of the second ECG sensing electrode 148).

The signal data 616, 618 generated by the ECG sensing electrodes 140, 148 and transmitted to the touch controller 104 is stored in the database 604. The signal data 616, 618 from the respective ECG sensing electrodes 140, 148 is processed by the A/D converter 136. In some examples, the signal data 616, 618 from the ECG sensing electrode(s) 140, 148 is filtered by the filter 606 to remove noise from the signal data 616, 618. For example, the signal data 616, 618 generated by the respective ECG sensing electrode(s) 140, 148 can include noise in the form of motion artifacts from user movement(s). The filter 606 can filter the signal data 616, 618 to remove the noise for improved identification of the ECG signal data.

The example digital signal processor 138 includes a calculator 620, or means for determining ECG signal data for the user. The means for determining ECG signal data may be implemented by a processor such as the processor of FIG. 8 executing instructions such as the instructions of FIG. 7. The example calculator 620 determines the ECG signal for the user from the first signal data 616 generated by the first ECG sensing electrode 140 and the second signal data 618 generated by the second ECG sensing electrode 148 indicative of capacitance changes at the drive line(s) 106 of the drive line set(s) 142, 150. In particular, the calculator 620 calculates the difference between the first signal data 616 and the second signal data 618 to generate ECG signal data 622 for the user.

The ECG signal data 622 is stored in the database 604. In some examples, the calculator 620 outputs the ECG signal data 622 for analysis with respect to user health metrics. In some examples, at least some of the analysis of the ECG signal data 622 is performed by the digital signal processor 138. For example, the example digital signal processor 138 of FIG. 6 includes an ECG data analyzer 624, or means for analyzing the ECG signal data 622. The means for analyzing may be implemented by a processor such as the processor of FIG. 8 executing instructions such as the instructions of FIG. 7. The example ECG data analyzer 624 can calculate metrics such as, for instance, a heart rate of the user based on the ECG signal data 622. In some examples, the ECG data analyzer 624 provides for analysis of the ECG signal data locally in examples in which at least a part of the digital signal processor 138 is implemented by an on-board processor of the personal computing device (e.g., by the on-board processor 401).

In some examples, the calculator 620 transmits the ECG signal data 622 to one or more ECG user applications 626 implemented by one or more processors of the personal computing device 400 and/or in a cloud-computing environment (e.g., via one or more wired or wireless communication connections). For example, the ECG user application(s) 626 may generate graphs displaying the ECG signal data, track historical ECG data over time, generate audio and/or visual alert(s) to be output the subject or other individuals (e.g., a doctor) via one or more user devices (e.g., the personal computing device 400). In some examples, the ECG data analyzer 624 transmits the ECG signal data 622 to the ECG user application(s) based on, for instance, processing resources to analyze the ECG signal data 622 with respect to particular characteristics to be analyzed. For example, the ECG data analyzer 624 may calculate the user's heart rate based on the ECG signal data 622, which the ECG user application(s) 626 generate graph(s) and track the data over time.

In some examples, the ECG data analyzer 624 and/or the ECG user application(s) 626 generate user interface graphics that are displayed via the display screen 102 and that provide visual indications to the user of where the user should place his or her body parts (e.g., thumbs, palms) on the display screen 102 for measurement of the user's ECG data. In such examples, the ECG electrode controller 612 activates the first and second ECG sensing electrodes 140, 148 when, for example, the touch event analyzer 608 determines that the user has touched the display screen 102 proximate to the ECG visual indicator regions and based on the ECG activator rule(s) 614 defining a length of time that the user is in contact with the display screen 102. In some examples, the ECG activator rule(s) 614 indicate that the ECG sensing electrodes 140, 148 should be activated when a user selects to run an ECG user application 626. In such examples, the execution of the ECG user application(s) 626 can serve as the trigger event for the activation of the ECG sensing electrodes 140, 148 by the ECG electrode controller 612.

After the ECG signal data 622 has been generated, the example touch controller 104 of FIG. 1 automatically instructs the display screen 102 to resume operating as a touch screen. In particular, based on the ECG activator rule(s) 614, the example ECG electrode controller 612 communicates with the drive signal activator 600 to cause the drive signal source 118 to resume generating drive signals that are transmitted to the drive lines 106 and measured by the sense line(s) 108 to detect user touch event(s). For example, the ECG activator rule(s) 614 can indicate that if no signal data is received by the touch controller 104 from the ECG sensing electrode(s) 140, 148 for a predefined period of time, the user has removed one or both of his or her hands from touching the display screen 102 and, thus, the drive signal source 118 should resume generating and transmitting drive signals to the drive lines 106 of the display screen 102. As another example, the ECG activator rule(s) 614 can indicate that the drive signal source 118 should resume generating and transmitting drive signals to the drive lines 106 after a predefined period of time, which can include a reference or threshold time for the drive lines 106 of the ECG sensing electrodes 140, 148 to detect a change in self-capacitance. The ECG activator rule(s) 614 can indicate that the drive signal source 118 should resume generating and transmitting drive signals to the drive lines 106 based on feedback from the ECG data analyzer 624 and/or the ECG user application(s) 626 indicating that the ECG signal data 622 has been received and/or that output(s) based on the ECG signal data 622 have been generated.

While an example manner of implementing the touch controller 104 is illustrated in FIGS. 1, 2, and 6, one or more of the elements, processes and/or devices illustrated in FIGS. 1, 2, and/or 6 may be combined, divided, re-arranged, omitted, eliminated and/or implemented in any other way. Further, the example multiplexer 130, the example integrator 134, the example A/D converter 136, the example digital signal processor 138, the example drive signal activator 600, the example database 604, the example filter 606, the example touch event analyzer 608, the example ECG electrode controller 612, the example calculator 620, the example ECG data analyzer 624 and/or, more generally, the example touch controller 104 of FIGS. 1, 2, and/or 6 may be implemented by hardware, software, firmware and/or any combination of hardware, software and/or firmware. Thus, for example, any of the example multiplexer 130, the example integrator 134, the example A/D converter 136, the example digital signal processor 138, the example drive signal activator 600, the example database 604, the example filter 606, the example touch event analyzer 608, the example ECG electrode controller 612, the example calculator 620, the example ECG data analyzer 624 and/or, more generally, the example touch controller 104 could be implemented by one or more analog or digital circuit(s), logic circuits, programmable processor(s), programmable controller(s), graphics processing unit(s) (GPU(s)), digital signal processor(s) (DSP(s)), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the example multiplexer 130, the example integrator 134, the example A/D converter 136, the example digital signal processor 138, the example drive signal activator 600, the example database 604, the example filter 606, the example touch event analyzer 608, the example ECG electrode controller 612, the example calculator 620, and/or the example ECG data analyzer 624 is/are hereby expressly defined to include a non-transitory computer readable storage device or storage disk such as a memory, a digital versatile disk (DVD), a compact disk (CD), a Blu-ray disk, etc. including the software and/or firmware. Further still, the example touch controller 104 of FIGS. 1, 2, and/or 6 may include one or more elements, processes and/or devices in addition to, or instead of, those illustrated in FIGS. 1, 2, and/or 6, and/or may include more than one of any or all of the illustrated elements, processes and devices. As used herein, the phrase “in communication,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events.

A flowchart representative of example hardware logic, machine readable instructions, hardware implemented state machines, and/or any combination thereof for implementing the touch controller 104 is shown in FIG. 7. The machine readable instructions may be one or more executable programs or portion(s) of an executable program for execution by a computer processor such as the processor 812 shown in the example processor platform 800 discussed below in connection with FIG. 8. The program may be embodied in software stored on a non-transitory computer readable storage medium such as a CD-ROM, a floppy disk, a hard drive, a DVD, a Blu-ray disk, or a memory associated with the processor 812, but the entire program and/or parts thereof could alternatively be executed by a device other than the processor 812 and/or embodied in firmware or dedicated hardware. Further, although the example program is described with reference to the flowchart illustrated in FIG. 7, many other methods of implementing the example the touch controller 104 may alternatively be used. For example, the order of execution of the blocks may be changed, and/or some of the blocks described may be changed, eliminated, or combined. Additionally or alternatively, any or all of the blocks may be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

The machine readable instructions described herein may be stored in one or more of a compressed format, an encrypted format, a fragmented format, a packaged format, etc. Machine readable instructions as described herein may be stored as data (e.g., portions of instructions, code, representations of code, etc.) that may be utilized to create, manufacture, and/or produce machine executable instructions. For example, the machine readable instructions may be fragmented and stored on one or more storage devices and/or computing devices (e.g., servers). The machine readable instructions may require one or more of installation, modification, adaptation, updating, combining, supplementing, configuring, decryption, decompression, unpacking, distribution, reassignment, etc. in order to make them directly readable and/or executable by a computing device and/or other machine. For example, the machine readable instructions may be stored in multiple parts, which are individually compressed, encrypted, and stored on separate computing devices, wherein the parts when decrypted, decompressed, and combined form a set of executable instructions that implement a program such as that described herein. In another example, the machine readable instructions may be stored in a state in which they may be read by a computer, but require addition of a library (e.g., a dynamic link library (DLL)), a software development kit (SDK), an application programming interface (API), etc. in order to execute the instructions on a particular computing device or other device. In another example, the machine readable instructions may need to be configured (e.g., settings stored, data input, network addresses recorded, etc.) before the machine readable instructions and/or the corresponding program(s) can be executed in whole or in part. Thus, the disclosed machine readable instructions and/or corresponding program(s) are intended to encompass such machine readable instructions and/or program(s) regardless of the particular format or state of the machine readable instructions and/or program(s) when stored or otherwise at rest or in transit.

As mentioned above, the example process of FIG. 7 may be implemented using executable instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media.

FIG. 7 is a flowchart of example machine readable instructions that, when executed, implement the example touch controller 104 of FIGS. 1, 2, and/or 6. In the example of FIG. 7, the touch controller 104 selectively controls operation of the display screen 102 of FIGS. 1 and 2 to operate in a first mode as a touch screen or in a second mode as an ECG sensor. The example instructions of FIG. 7 can be executed by one or more processors of, for instance, a user device including the display screen 102 (e.g., the personal computing device 400) and/or a cloud-based device. The instructions of FIG. 7 can be executed in substantially real-time as, for example, signal data indicative of user touch gestures on the display screen 102 is received by the controller 104. In some examples, some of the instructions of FIG. 7 are executed some time after, for instance, signal data associated with the user's ECG data is received by the touch controller 104.

The example drive signal activator 600 of FIG. 6 instructs the drive signal source 118 (e.g., a generator) to generate drive signals that are transmitted to the drives lines 106 of the display screen 102 (block 700). The drive signal activator 600 instructs the drive signal source to generate the drive signals based on drive signal rule(s) 602. The drive signal rule(s) 602 can be stored in the database 604.

The multiplexer 130 of FIGS. 1, 2, and 6 accesses signal data generated by the sense line(s) 108 of the display screen 102 (block 702). As discussed above, the sense line(s) 108 detect voltage drops with respect to the current flowing through the drive line(s) 106 when a user touches the display screen 102 due to changes in mutual capacitance. The multiplexer 130 receives signal data 605 from the sense line(s) 108 indicative of the voltage changes and provides the data to the integrator 134 of the touch controller 104. The integrator 134 performs an integration on the signal data 605 from the sense line(s) 108 and transmits the signal data the A/D converter 136 of the touch controller 104 for digital processing.

The example touch event analyzer 608 of the digital signal processor of FIG. 6 analyzes the signal data 605 from the sense line(s) 108 to determine if the signal data 605 indicates a user touch gesture on the display screen 102 (block 704). For example, the touch event analyzer 608 identifies touch events based on the touch event rule(s) 610 that define, for instance, threshold voltage drop values corresponding to user touch event(s) and location(s) of the touch event(s).

In the example of FIG. 7, if the touch event analyzer 608 identifies a user touch event, the ECG electrode controller 612 of FIG. 6 determines if the touch event represents a trigger event to activate the ECG sensing electrodes 140, 148 (block 706). For example, based on the ECG activator rule(s) 614 and the data received from the touch event analyzer 608, the ECG electrode controller 612 determines if the user has touched the display screen 102 proximate to the areas of the display screen 102 including the ECG sensing electrodes 140, 148 (e.g., proximate to the third and fourth edges 114, 116 of the display screen 102). In some examples, the ECG electrode controller 612 determines if the user has touched the display screen 102 proximate to the areas of the display screen 102 including the ECG sensing electrodes 140, 148 for a predetermined period of time based on the ECG activator rule(s) 614 and data received from the touch event analyzer 608.

In the example of FIG. 7, if the touch event analyzer 608 does not detect a touch event based on the signal data from the sense line(s) 108 or if the touch event analyzer detects a touch event, but does not detect an ECG trigger event based on the touch event, then the touch controller 104 continues to access signal data from the sense line(s) 108 of the display screen 102 (block 702) and the touch event analyzer 608 continues to analyze the signal data to detect touch event(s) (blocks 704, 706).

If the ECG electrode controller 612 determines that an ECG trigger event has occurred, the ECG electrode controller 612 communicates with the drive signal activator 600 to instruct the drive signal source 118 to refrain from generating the drive signals (block 708). The ECG electrode controller 612 also activates the first and second ECG sensing electrodes 140, 148 (block 710). For example, the ECG electrode controller 612 generates instructions for data from the drive line(s) 106 to be transmitted to the integrators 144, 152 and the amplifiers 146, 154 of the respective ECG sensing electrodes 140, 148. As discussed above, when the first and second ECG sensing electrodes 140, 148 are activated, the touch functionality of the display screen 102 is disabled due to the lack of drive signals from the drive signal source 118.

When the display screen 102 is operating in the ECG-sensing mode, the A/D converter 136 of the touch controller 104 accesses first signal data 616 generated by the first ECG sensing electrode 140 in response to changes in self-capacitance detected by the drive line(s) 106 of the first drive line set 142 of the first ECG sensing electrode 140 (block 712). The A/D converter 136 accesses second signal data 618 generated by the second ECG sensing electrode 148 in response to changes in self-capacitance detected by the drive line(s) 106 of the second drive line set 150 of the second ECG sensing electrode 148 (block 712). The A/D converter 136 converts the signal data 616, 618 to digital data for processing by the digital signal processor 138. In some examples, the filter 606 filters the signal date for noise related to, for instance, user movements.

The calculator 620 of FIG. 6 generates ECG signal data 622 by calculating the difference between the first signal data 616 from the first ECG sensing electrode 140 and the second signal data 618 from the second ECG sensing electrode 148 (block 714). The ECG signal data 622 can be stored in the database 604.

The calculator 620 of FIG. 6 outputs the ECG signal data for analysis with respect to, for example, health metrics of the user (block 716). In some examples, the ECG data analyzer 624 of the digital signal processor 138 performs at least some analysis of the ECG signal data. For instance, the ECG data analyzer 624 can calculate the user's heart rate based on the ECG signal data 622. In some examples, the calculator 620 transmits the ECG signal data 622 to one or more ECG user applications 626 implemented by processor(s) of the personal computing device including the display screen 102 and/or by cloud-based device(s).

In FIG. 7, the ECG electrode controller 612 determines if additional ECG data should be collected from the user and, thus, the ECG sensing electrodes 140, 148 should remain active, or if the display screen 102 should return to operating as a touch screen (block 718). If additional ECG signal data is to be collected, then the example of FIG. 7 continues to access signal data generated by the first and second ECG sensing electrodes 140, 148 (block 712). However, if no additional ECG signal data is to be collected, then the example of FIG. 7 determines that the display screen 102 should return to operating as a touch screen. For example, if the A/D converter 136 does not receive signal data 616, 618 from the ECG sensing electrode(s) 140, 148 for a predefined period of time, the ECG electrode controller 612 determines that the user removed his or her hand(s) from the display screen. In such examples, the ECG electrode controller 612 generates instructions for the integrators 144, 152 and the amplifiers 146, 154 of the respective ECG sensing electrodes 140, 148 to become inactive.

When no additional ECG data is to be collected, the ECG electrode controller 612 communicates with the drive signal activator 600 to instruct the drive signal source 118 to resume generation and transmission of drive signals to the drive line(s) 106 of the display screen 102, thereby reactivating the touch functionality of the display screen 102 (block 720). The example instructions of FIG. 7 end when the display screen 102 is powered off (block 722).

FIG. 8 is a block diagram of an example processor platform 800 structured to execute the instructions of FIG. 7 to implement the example touch controller 104 of FIGS. 1, 2, and/or 6. The processor platform 800 can be, for example, a server, a personal computer, a workstation, a self-learning machine (e.g., a neural network), a mobile device (e.g., a cell phone, a smart phone, a tablet such as an iPad™), a personal digital assistant (PDA), an Internet appliance, a headset or other wearable device, or any other type of computing device.

The processor platform 800 of the illustrated example includes a processor 104. The processor 104 of the illustrated example is hardware. For example, the processor 104 can be implemented by one or more integrated circuits, logic circuits, microprocessors, GPUs, DSPs, or controllers from any desired family or manufacturer. The hardware processor may be a semiconductor based (e.g., silicon based) device. In this example, the processor executes the instructions represented by FIG. 7 to implement the example multiplexer 130, the example integrator 134, the example A/D converter 136, the example digital signal processor 138, the example drive signal activator 600, the example database 604, the example filter 606, the example touch event analyzer 608, the example ECG electrode controller 612, the example calculator 620, and/or the example ECG data analyzer 624.

The processor 104 of the illustrated example includes a local memory 813 (e.g., a cache). The processor 812 of the illustrated example is in communication with a main memory including a volatile memory 814 and a non-volatile memory 816 via a bus 818. The volatile memory 814 may be implemented by Synchronous Dynamic Random Access Memory (SDRAM), Dynamic Random Access Memory (DRAM), RAMBUS® Dynamic Random Access Memory (RDRAM®) and/or any other type of random access memory device. The non-volatile memory 816 may be implemented by flash memory and/or any other desired type of memory device. Access to the main memory 814, 816 is controlled by a memory controller.

The processor platform 800 of the illustrated example also includes an interface circuit 820. The interface circuit 820 may be implemented by any type of interface standard, such as an Ethernet interface, a universal serial bus (USB), a Bluetooth® interface, a near field communication (NFC) interface, and/or a PCI express interface.

In the illustrated example, one or more input devices 822 are connected to the interface circuit 820. The input device(s) 822 permit(s) a user to enter data and/or commands into the processor 812. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 824 are also connected to the interface circuit 820 of the illustrated example. The output devices 824 can be implemented, for example, by display devices (e.g., a light emitting diode (LED), an organic light emitting diode (OLED), a liquid crystal display (LCD), a cathode ray tube display (CRT), an in-place switching (IPS) display, a touchscreen, etc.), a tactile output device, a printer and/or speaker. The interface circuit 820 of the illustrated example, thus, typically includes a graphics driver card, a graphics driver chip and/or a graphics driver processor.

The interface circuit 820 of the illustrated example also includes a communication device such as a transmitter, a receiver, a transceiver, a modem, a residential gateway, a wireless access point, and/or a network interface to facilitate exchange of data with external machines (e.g., computing devices of any kind) via a network 826. The communication can be via, for example, an Ethernet connection, a digital subscriber line (DSL) connection, a telephone line connection, a coaxial cable system, a satellite system, a line-of-site wireless system, a cellular telephone system, etc.

The processor platform 800 of the illustrated example also includes one or more mass storage devices 828 for storing software and/or data. Examples of such mass storage devices 828 include floppy disk drives, hard drive disks, compact disk drives, Blu-ray disk drives, redundant array of independent disks (RAID) systems, and digital versatile disk (DVD) drives.

The machine executable instructions 832 of FIG. 7 may be stored in the mass storage device 828, in the volatile memory 814, in the non-volatile memory 816, and/or on a removable non-transitory computer readable storage medium such as a CD or DVD.

From the foregoing, it will be appreciated that example apparatus. methods, and articles of manufacture have been disclosed that provide for sensing of ECG data from a user via an electronic touch display screen of a personal computing device. Examples disclosed herein provide for selective operation of the display screen in a first mode as a touch screen to receive user touch inputs to enable the user to interact with digital content presented via the display screen or in a second mode in which the display screen as an ECG sensor. In examples disclosed herein, drive lines of the display screen can detect changes in self-capacitance when the user touches the display screen and, thus, act as capacitive ECG electrodes. Accordingly, example display screens disclosed herein utilize existing components of the display screen to sense user ECG data that can provide indications of the user's health.

Example 1 includes an electronic user device including a display screen including a first electrode and a second electrode and a processor operatively coupled to the display screen. The processor is to cause the display screen to operate in a first display screen mode to detect a touch input from a user on the display screen. The processor is to cause the display screen to switch from operating in the first display screen mode to operating in a second display screen mode. The first electrode and the second electrode are to generate signal data indicative of electrocardiogram data for the user when the display screen is operating in the second display screen mode.

Example 2 includes the electronic user device as defined in example 1, wherein the processor is to cause the display screen to operate in the second display screen mode based on the touch input from the user.

Example 3 includes the electronic user device as defined in example 1, wherein the display screen includes a plurality of drive lines and the first electrode includes at least one of the plurality of drive lines.

Example 4 includes the electronic user device as defined in example 3, wherein the first electrode further includes an amplifier in communication with the at least one of the plurality of drive lines.

Example 5 includes the electronic user device as defined in example 3, wherein the at least one of the plurality of drive lines is to transmit a drive signal when the display screen is operating in the first display screen mode and generate signal indicative of a change in capacitance when the display screen is operating in the second display screen mode.

Example 6 includes the electronic user device as defined in examples 1 or 2, wherein the first electrode is to generate first signal data in response to a first touch of the user on the display screen proximate to the first electrode and the second electrode is to generate second signal data in response to a second touch of the user on the display screen proximate to the second electrode when the display screen is operating in the second display screen mode and the processor is to calculate a difference between the first signal data and the second signal data and output electrocardiogram signal data for the user based on the difference.

Example 7 includes the electronic user device as defined in example 1, wherein the first electrode is disposed proximate to a first edge of the display screen and the second electrode is disposed proximate to a second edge of the display screen opposite the first edge.

Example 8 includes the electronic user device as defined in example 7, wherein the processor is to detect a first touch input by the user proximate to the first edge of the display screen, detect a second touch input by the user proximate to the second edge of the display screen, and cause the display screen to switch from operating in the first display screen mode to operating in the second display screen mode based on the first touch input and the second touch input.

Example 9 includes the electronic user device as defined in examples 1 or 2, wherein the display screen is a first display screen and further including a second display screen, the second display screen to operate in the first display screen mode when the first display screen is operating in the second display screen mode.

Example 10 includes an apparatus including a display screen including drive lines to transmit drive signals, sense lines, a first electrode including a first portion of the drive lines, and a touch controller operatively coupled to the display screen. The touch controller to cause the drive lines to transmit drive signals, the sense lines to detect the drive signals, detect a touch event by a user on the display screen based on signal data received from the sense lines, and activate the first electrode based on the touch event, the first portion of the drive lines to generate first signal data indicative of electrocardiogram data for the user when the first electrode is activated.

Example 11 includes the apparatus of example 10, wherein the display screen includes a second electrode including a second portion of the drive lines, the touch controller to further activate the second electrode based on the touch event, the second portion of the drive lines to generate second signal data indicative of the electrocardiogram data for the user when the second electrode is activated.

Example 12 includes the apparatus of example 11, wherein the touch controller is to generate electrocardiogram data for the user based the first signal data and the second signal data.

Example 13 includes the apparatus of example 12, wherein the touch controller is to cause the first portion of the drives lines to switch from generating the first signal data to transmitting the drive signals based on the generation of the electrocardiogram data.

Example 14 includes the apparatus of example 11, wherein the first electrode is disposed at a first region of the display screen and the second electrode is disposed at a second region of the display screen and the touch event includes a first touch input received at the first region and a second touch input received at the second region.

Example 15 includes the apparatus of example 11, wherein the first electrode is disposed at a first region of the display screen and the second electrode is disposed at a second region of the display screen and the touch event includes a first touch input received at the first region and a second touch input received at the second region.

Example 16 includes the apparatus of example 10, wherein the first electrode further includes an integrator and an amplifier.

Example 17 include at least one non-transitory computer readable storage medium comprising instructions that, when executed, cause a machine to cause a display screen of an electronic user device to operate in a first mode, the display screen including drives lines to transmit drive signals and sense lines to generate signal data indicative of a touch of a user on the display screen based on the drive signals when the display screen is operating the first mode and cause the display screen to switch from operating in the first mode to operating in a second mode, the display screen including an electrode to generate signal data indicative of electrocardiogram data for the user when the display screen is operating in the second mode.

Example 18 includes the at least one non-transitory computer readable storage medium as defined in example 17, wherein the instructions, when executed, further cause the machine to selectively instruct a drive signal generator to generate the drive signals based on the operation of the display screen in the first mode or the second mode.

Example 19 includes the at least one non-transitory computer readable storage medium as defined in example 17, wherein the electrode includes a first electrode and a second electrode, the signal data includes first signal data generated by the first electrode and second signal data generated by the second electrode, and the instructions, when executed, further cause the machine to calculate electrocardiogram signal data for the user based on the first signal data and the second signal data.

Example 20 includes the at least one non-transitory computer readable storage medium as defined in example 19, wherein the instructions, when executed, further cause the machine to determine a health metric for the user based on the electrocardiogram signal data.

Example 21 includes the at least one non-transitory computer readable storage medium as defined in example 17, wherein the instructions, when executed, further cause the machine to cause the display screen to operate in the second mode based on the signal data from the sense lines.

Example 22 includes the at least one non-transitory computer readable storage medium as defined in example 17, wherein the instructions further cause the machine to filter the signal data generated by the electrode.

Example 23 includes an apparatus comprising a display screen including first means for sensing electrocardiogram data from a subject, second means for sensing electrocardiogram data from a subject, means for causing the display screen to operate in a first mode as a touch screen, and means for selectively activating the first means for sensing and the second means for sensing to cause the display screen to operate in a second mode as an electrocardiogram sensor.

Example 24 includes the apparatus of example 23, wherein the means for causing the display screen to operate in a first mode is to cause a drive signal generator to transmit drive signals to drive lines of the display screen.

Example 25 includes the apparatus of example 23, wherein the means for selectively activating is to activate the first means for sensing and the second means for sensing based on a touch input on the display screen.

Example 26 includes the apparatus of example 23, further including means for determining electrocardiogram signal data for the subject based on first signal data from the first means for sensing and second signal data from the second means for sensing.

Example 27 includes the apparatus of example 26, further including means for analyzing the electrocardiogram signal data to determine a health metric for the subject.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc. may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, and (7) A with B and with C. As used herein in the context of describing structures, components, items, objects and/or things, the phrase “at least one of A and B” is intended to refer to implementations including any of (1) at least one A, (2) at least one B, and (3) at least one A and at least one B. Similarly, as used herein in the context of describing structures, components, items, objects and/or things, the phrase “at least one of A or B” is intended to refer to implementations including any of (1) at least one A, (2) at least one B, and (3) at least one A and at least one B. As used herein in the context of describing the performance or execution of processes, instructions, actions, activities and/or steps, the phrase “at least one of A and B” is intended to refer to implementations including any of (1) at least one A, (2) at least one B, and (3) at least one A and at least one B. Similarly, as used herein in the context of describing the performance or execution of processes, instructions, actions, activities and/or steps, the phrase “at least one of A or B” is intended to refer to implementations including any of (1) at least one A, (2) at least one B, and (3) at least one A and at least one B.

Although certain example methods, apparatus and articles of manufacture have been disclosed herein, the scope of coverage of this patent is not limited thereto. On the contrary, this patent covers all methods, apparatus and articles of manufacture fairly falling within the scope of the claims of this patent.

1. An electronic user device comprising:
a display screen including a first electrode and a second electrode; and
a processor operatively coupled to the display screen, the processor to:
cause the display screen to operate in a first display screen mode to detect a touch input from a user on the display screen; and
cause the display screen to switch from operating in the first display screen mode to operating in a second display screen mode, the first electrode and the second electrode to generate signal data indicative of electrocardiogram data for the user when the display screen is operating in the second display screen mode.

2. The electronic user device as defined in claim 1, wherein the processor is to cause the display screen to operate in the second display screen mode based on the touch input from the user.

3. The electronic user device as defined in claim 1, wherein the display screen includes a plurality of drive lines and the first electrode includes at least one of the plurality of drive lines.

4. The electronic user device as defined in claim 3, wherein the first electrode further includes an amplifier in communication with the at least one of the plurality of drive lines.

5. The electronic user device as defined in claim 3, wherein the at least one of the plurality of drive lines is to:
transmit a drive signal when the display screen is operating in the first display screen mode; and
generate signal indicative of a change in capacitance when the display screen is operating in the second display screen mode.

6. The electronic user device as defined in claim 1, wherein the first electrode is to generate first signal data in response to a first touch of the user on the display screen proximate to the first electrode and the second electrode is to generate second signal data in response to a second touch of the user on the display screen proximate to the second electrode when the display screen is operating in the second display screen mode, the processor to:
calculate a difference between the first signal data and the second signal data; and
output electrocardiogram signal data for the user based on the difference.

7. The electronic user device as defined in claim 1, wherein the first electrode is disposed proximate to a first edge of the display screen and the second electrode is disposed proximate to a second edge of the display screen opposite the first edge.

8. The electronic user device as defined in claim 7, wherein the processor is to:
detect a first touch input by the user proximate to the first edge of the display screen;
detect a second touch input by the user proximate to the second edge of the display screen; and
cause the display screen to switch from operating in the first display screen mode to operating in the second display screen mode based on the first touch input and the second touch input.

9. The electronic user device as defined in claim 1, wherein the display screen is a first display screen and further including a second display screen, the second display screen to operate in the first display screen mode when the first display screen is operating in the second display screen mode.

10. An apparatus comprising:
a display screen including:
drive lines to transmit drive signals;
sense lines;
a first electrode including a first portion of the drive lines; and
a touch controller operatively coupled to the display screen, the touch controller to:
cause the drive lines to transmit drive signals, the sense lines to detect the drive signals;
detect a touch event by a user on the display screen based on signal data received from the sense lines; and
activate the first electrode based on the touch event, the first portion of the drive lines to generate first signal data indicative of electrocardiogram data for the user when the first electrode is activated.

11. The apparatus of claim 10, wherein the display screen includes a second electrode including a second portion of the drive lines, the touch controller to further activate the second electrode based on the touch event, the second portion of the drive lines to generate second signal data indicative of the electrocardiogram data for the user when the second electrode is activated.

12. The apparatus of claim 11, wherein the touch controller is to generate electrocardiogram data for the user based the first signal data and the second signal data.

13. The apparatus of claim 12, wherein the touch controller is to cause the first portion of the drives lines to switch from generating the first signal data to transmitting the drive signals based on the generation of the electrocardiogram data.

14. The apparatus of claim 11, wherein the first electrode is disposed at a first region of the display screen and the second electrode is disposed at a second region of the display screen and the touch event includes a first touch input received at the first region and a second touch input received at the second region.

15. The apparatus of claim 10, further including a drive signal generator in communication with the touch controller, the touch controller to selectively cause the drive signal generator to generate the drive signals based on the signal data from the sense lines.

16. The apparatus of claim 10, wherein the first electrode further includes an integrator and an amplifier.

17. At least one non-transitory computer readable storage medium comprising instructions that, when executed, cause a machine to:
cause a display screen of an electronic user device to operate in a first mode, the display screen including drives lines to transmit drive signals and sense lines to generate signal data indicative of a touch of a user on the display screen based on the drive signals when the display screen is operating the first mode; and
cause the display screen to switch from operating in the first mode to operating in a second mode, the display screen including an electrode to generate signal data indicative of electrocardiogram data for the user when the display screen is operating in the second mode.

18. The at least one non-transitory computer readable storage medium as defined in claim 17, wherein the instructions, when executed, further cause the machine to selectively instruct a drive signal generator to generate the drive signals based on the operation of the display screen in the first mode or the second mode.

19. The at least one non-transitory computer readable storage medium as defined in claim 17, wherein the electrode includes a first electrode and a second electrode, the signal data includes first signal data generated by the first electrode and second signal data generated by the second electrode, and the instructions, when executed, further cause the machine to calculate electrocardiogram signal data for the user based on the first signal data and the second signal data.

20. (canceled)

21. The at least one non-transitory computer readable storage medium as defined in claim 17, wherein the instructions, when executed, further cause the machine to cause the display screen to operate in the second mode based on the signal data from the sense lines.

22.-27. (canceled)
